(19) United States
(12) Patent Application Publication
(10) Pub. No.: US 2005/0244298 A1
(43) Pub. Date: Nov. 3, 2005

(54) Electrocardiographic aspects of chf treatment

(76) Inventors: Eldon R Smith, Calgary, Alberta (CA); Guillermo Torre-Amione, Bellaire, TX (US)

Correspondence Address: FOLEY & LARDNER LLP, 1530 PAGE MILL ROAD, PALO ALTO, CA, 94304, US

(21) Appl. No.: 10/510,594

(22) PCT Filed: Apr. 10, 2003
(86) PCT No.: PCT/CA03/00538

§ 371 Date: May 24, 2005

Related U.S. Application Data
(60) Provisional application No. 60/371,153, filed on Apr. 10, 2002.

(51) Int. Cl. A61M 1/14

(52) U.S. Cl. 422/45

(57) ABSTRACT

In accordance with the present invention, it has been found that prolonged QT-c intervals in mammalian patients, indicative of susceptibility of the patients to arrhythmia and sudden cardiac death, can be reduced by a process in which an aliquot of the patient's blood is removed and stressed extracorporeally, by application thereto of oxidative stress and electromagnetic radiation such as ultraviolet light, and then re-injected into the patient. Such treatment results in a significant reduction in QT-c interval in the patients, indicative of reduced susceptibility to arrhythmia and sudden cardiac death. In clinical trials described in the Examples section below, this reduction in QT-c interval was associated with a marked reduction in sudden cardiac death. There are also indications that, in the absence of treatment according to the invention, the patients would have exhibited a lengthening of their QT-c intervals.

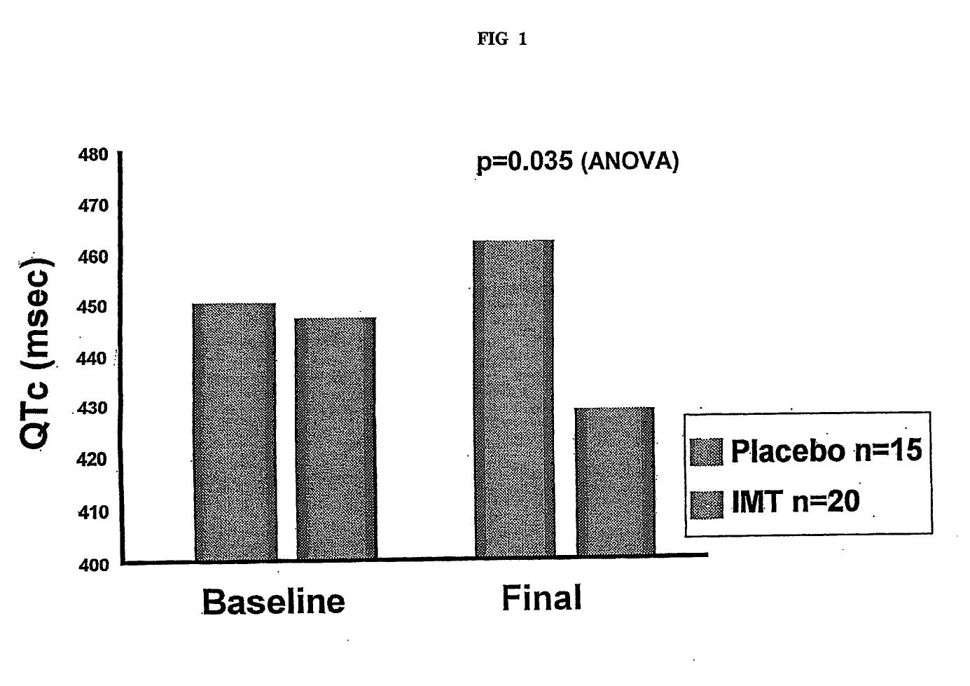

### FIELD OF THE INVENTION

This invention relates to medical treatments, compositions useful in medical treatments, and methods of preparation and use of such compositions. More specifically, it relates to treatments and compositions for reducing the risk and incidence of cardiac arrhythmia and sudden death from heart disease, in mammalian patients.

### BACKGROUND OF THE INVENTION

Of the approximately one million deaths from cardiovascular disease in North America annually, about one half result from ventricular arrhythmia (ventricular tachycardia or fibrillation), and are defined as “sudden”. Identification of patients at high risk of developing such malignant arrythmias, so that effective preventative strategies can be applied, is a major challenge to healthcare providers.

Serious ventricular arrhythmias develop because of inhomogeneities in the electrical properties of heart cells. Heart cells contract, to provide the necessary pumping action of the heart, because of a wave of electricity that spreads over the heart muscle—referred to as depolarization. Before the cells can contract again, for the next beat, they must recover their resting state—referred to as re-polarization. It is generally acknowledged that serious ventricular arrhythmias develop because of non-homogeneous recovery of the repolarization in the heart cells. Thus, contiguous areas of the heart muscle can have different durations of electrical activity. If one area is completely recovered (repolarized), and is therefore capable of being stimulated again, while adjacent areas are still partially depolarized, there is the potential for current flow from the area with prolonged recovery (partially depolarized) to a zone that is totally recovered. This current flow (re-entry) can result in a premature complex, and, under some conditions, result in a sustained ventricular tachycardia.

Variation in cardiac electrical recovery times from different areas of the heart is reflected in the QT interval on the electrocardiogram (ECG) of the heart. The QT interval represents the total duration of the electrical activity of the heart during each heart beat. It is usually corrected e.g. using Bazett's formula or the Fridericia formula to compensate for its known dependence on heart rate, to give QT-c. Normal value of QT-c is up to about 0.44 seconds. Abnormalities in the heart muscle, which result in decreased homogeneity of electrical activity may lead to instability, arrhythmias and sudden death, are often detectable as prolongation of the QT interval.

Another way of expressing inhomogeneity of electrical activity of the hearet is QT dispersion, QTd, which denotes the variability of the QT interval, from the shortest QT interval to the longest QT interval determined at different locations of the beating heart from the electrocardiogram leads.

It is an object of the present invention to provide a method of reducing QT intervals in mammalian patients exhibiting prolonged QT intervals, and thereby to reduce the risk of sudden cardiac death in such patients.

It is a further object of the invention to provide a method and composition for preventing, reducing the incidence or severity of, or treating, arrhythmias in mammalian patients.

It is a further and more general object of the present invention to provide a process of treatment or prophylaxis of any medical disorder, the presence of or susceptibility to which, in a mammalian patient, is indicated by a prolonged QT interval, or with which prolonged QT interval is associated.

### SUMMARY OF THE INVENTION

In accordance with the present invention, it has been found that prolonged QT-c intervals in mammalian patients, indicative of susceptibility of the patients to arrhythmia and sudden cardiac death, can be reduced by a process in which an aliquot of the patient's blood is removed and stressed extracorporeally, by application thereto of oxidative stress and electromagnetic radiation such as ultraviolet light, and then re-injected into the patient. Such treatment results in a significant reduction in QT-c interval in the patients, indicative of reduced susceptibility to arrhythmia and sudden cardiac death. In clinical trials described in the Examples section below, this reduction in QT-c interval was associated with a marked reduction in sudden cardiac death. There are also indications that, in the absence of treatment according to the invention, the patients would have exhibited a lengthening of their QT-c intervals.

According to the invention, the blood aliquot is modified extracorporeally by subjecting the blood, or separated cellular fractions of the blood, or mixtures of the separated cells and non-cellular fractions of the blood, to combinations of oxidative stressor and electromagnetic radiation, and optionally a heat stressor, simultaneously or sequentially.

### BRIEF REFERENCE TO THE DRAWINGS

The accompanying FIGURE of drawing is a bar graph of QT interval of patients before and after treatment according to the preferred process of the invention, as described in the specific example below.

### DETAILED DESCRIPTION OF PREFERRED EMBODIMENTS

According to a preferred process of the present invention, an aliquot of blood is extracted from a human subject, exhibiting prolonged QT interval, and the aliquot of blood is treated ex vivo with certain stressors, described in more detail below. The terms “aliquot”, “aliquot of blood” or similar terms used herein include whole blood, separated cellular fractions of the blood including platelets, separated non-cellular fractions of the blood including plasma, and combinations thereof. The effect of the stressors is to modify the blood, and/or the cellular or non-cellular fractions thereof, contained in the aliquot. The modified aliquot is then re-introduced into the subject's body by any route suitable for vaccination, preferably selected from intra-arterial injection, intramuscular injection, intravenous injection, subcutaneous injection, intraperitoneal injection, and oral, nasal or rectal administration, most preferably intramuscular injection.

The stressors to which the aliquot of blood is subjected ex vivo according to the method of the present invention are selected from temperature stress {blood temperature above or below body temperature), an oxidative environment and an electromagnetic emission, individually or in any combination, simultaneously or sequentially. Suitably, in human subjects, the aliquot has a volume sufficient that, when re-introduced into the subject's body, a significant reduction of the abnormal QT interval is achieved in the subject. Preferably, the volume of the aliquot is up to about 400 ml, preferably from about 0.1 to about 100 ml, more preferably from about 5 to about 15 ml, even more preferably from about 8 to about 12 ml, and most preferably about 10 ml, along with an anticoagulant, e.g. 2 ml sodium citrate.

It is preferred, according to the invention, to apply all three of the aforementioned stressors simultaneously to the aliquot under treatment, in order to ensure the appropriate modification to the blood. It may also be preferred in some embodiments of the invention to apply any two of the above stressors, for example to apply temperature stress and oxidative stress, temperature stress and an electromagnetic emission, or an electromagnetic 5 emission and oxidative stress. Care must be taken to utilize an appropriate level of the stressors to thereby effectively modify the blood to reduce QT interval in the subject.

The temperature stressor warms the aliquot being treated to a temperature above normal body temperature or cools the aliquot below normal body temperature. The temperature is selected so that the temperature stressor does not cause excessive hemolysis in the blood contained in the aliquot and so that, when the treated aliquot is injected into a subject, reduction of the abnormal QT interval will be achieved. Preferably, the temperature stressor is applied so that the temperature of all or a part of the aliquot is up to about 55° C., and more preferably in the range of from about −5° C. to about 55° C.

In some preferred embodiments of the invention, the temperature of the aliquot is raised above normal body temperature, such that the mean temperature of the aliquot does not exceed a temperature of about 55° C., more preferably from about 40° C. to about 50° C., even more preferably from about 40° C. to about 44° C., and most preferably about 42.5% ±1° C.

In other preferred embodiments, the aliquot is cooled below normal body temperature such that the mean temperature of the aliquot is within the range of from about −5° C. to about 36.5° C., more preferably from about 10° C. to about 30° C., and even more preferably from about 15° C. to about 25° C.

The oxidative environment stressor can be the application to the aliquot of solid, liquid or gaseous oxidizing agents. Preferably, it involves exposing the aliquot to a mixture of medical grade oxygen and ozone gas, most preferably by bubbling through the aliquot, at the aforementioned temperature range, a stream of medical grade oxygen gas having ozone as a minor component therein. The ozone content of the gas stream and the flow rate of the gas stream are preferably selected such that the amount of ozone introduced to the blood aliquot, either on its own or in combination with other stressors, does not give rise to excessive levels of cell damage such that the therapy is rendered ineffective. Suitably, the gas stream has an ozone content of up to about 300 μg/ml, preferably up to about 100 μg/ml, more preferably about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 litres/min, preferably up to about 0.5 litres/min, more preferably up to about 0.4 litres/min, even more preferably up to about 0.33 litres/min, and most preferably about 0.24±0.024 litres/min, at STP. The lower limit of the flow rate of the gas stream is preferably not lower than 0.01 litres/min, more preferably not lower than 0.1 litres/min, and even more preferably not lower than 0.2 litres/min.

The electromagnetic emission stressor is suitably applied by irradiating the aliquot under treatment from a source of an electromagnetic emission while the aliquot is maintained at the aforementioned temperature and while the oxygen/ozone gaseous mixture is being bubbled through the aliquot. Preferred electromagnetic emissions are selected from photonic radiation, more preferably UV, visible and infrared light, and even more preferably UV light. The most preferred UV sources are UV lamps emitting primarily UV-C band wavelengths, i.e. at wavelengths shorter than about 280 nm. Such lamps may also emit amounts of visible and infrared light. Ultraviolet light corresponding to standard UV-A (wavelengths from about 315 to about 400 nm) and UV-B (wavelengths from about 280 to about 315) sources can also be used. For example, an appropriate dosage of such UV light, applied simultaneously with the aforementioned temperature and oxidative environment stressors, can be obtained from lamps with a combined power output of from about 15 to about 25 watts, arranged to surround the sample container holding the aliquot, each lamp providing an intensity at a distance of 1 meter, of from about 45-65 mW/cm². Up to eight such lamps surrounding the sample bottle, with a combined output at 253.7 nm of 15-25 watts, operated at an intensity to deliver a total UV light energy at the surface of the blood of from about 0.025 to about 10 joules/cm², preferably from about 0.1 to about 3.0 joules/cm², may advantageously be used. Preferably, four such lamps are used.

The time for which the aliquot is subjected to the stressors is normally within the time range of up to about 60 minutes. The time depends to some extent upon the chosen intensity of the electromagnetic emission, the temperature, the concentration of the oxidizing agent and the rate at which it is supplied to the aliquot. Some experimentation to establish optimum times may be necessary on the part of the operator, once the other stressor levels have been set. Under most stressor conditions, preferred times will be in the approximate range of from about 2 to about 5 minutes, more preferably about 3 or about 3½% minutes. The starting blood temperature, and the rate at which it can be warmed or cooled to a predetermined temperature, tends to vary from subject to subject. Such a treatment provides a modified blood aliquot which is ready for injection into the subject.

In the practice of the preferred process of the present invention, the blood aliquot may be treated with the stressors using an apparatus of the type described in U.S. Pat. No. 4,968,483 to Mueller. The aliquot is placed in a suitable, sterile, UV light-transmissive container, which is fitted into the machine. The UV lamps are switched on for a fixed period before the gas flow is applied to the aliquot providing the oxidative stress, to allow the output of the UV lamps to stabilize. The UV lamps are typically on while the temperature of the aliquot is adjusted to the predetermined value, e.g. 42.5±1° C. Then the oxygen/ozone gas mixture, of known composition and controlled flow rate, is applied to the aliquot, for the predetermined duration of up to about 60 minutes, preferably 2 to 5 minutes and most preferably about 3 minutes as discussed above, so that the aliquot experiences all three stressors simultaneously. In this way, blood is appropriately modified according to the present invention to achieve the desired effects.

A subject preferably undergoes a course of treatments, each individual treatment comprising removal of a blood aliquot, treatment thereof as described above and re-administration of the treated aliquot to the subject. A course of such treatments may comprise daily administration of treated blood aliquots for a number of consecutive days, or may comprise a first course of daily treatments for a designated period of time, followed by an interval and then one or more additional courses of daily treatments.

In one preferred embodiment, the subject is given an initial course of treatments comprising the administration of 4 to 6 aliquots of treated blood. In another preferred embodiment, the subject is given an initial course of therapy comprising administration of from 2 to 4 aliquots of treated blood, with the administration of any pair of consecutive aliquots being either on consecutive days, or being separated by a rest period of from 1 to 21 days on which no aliquots are administered to the patient, the rest period separating one selected pair of consecutive aliquots being from about 3 to 15 days. In a more specific, preferred embodiment, the dosage regimen of the initial course of treatments comprises a total of three aliquots, with the first and second aliquots being administered on consecutive days and a rest period of 11 days being provided between the administration of the second and third aliquots.

It may be preferred to subsequently administer additional courses of treatments following the initial course of treatments. Preferably, subsequent courses of treatments are administered at least about three weeks after the end of the initial course of treatments. In one particularly preferred embodiment, the subject receives a second course of treatments comprising the administration of one aliquot of treated blood every 30 days following the end of the initial course of treatments, for a period of 6 months.

It will be appreciated that the spacing between successive courses of treatments should be such that the positive effects of the treatment of the invention are maintained, and may be determined on the basis of the observed response of individual subjects.

Many patients with cardiac disorders who are at risk of sudden cardiac death are administered various medications, comprising the current standard of care for such patients. The method of the present invention may preferably be used as an adjunctive treatment in combination with other therapies for sudden cardiac death or arrhythmia. Preferred examples of such other therapies include one or more of ACE inhibitors, β-blockers, aldosterone antagonists, digoxin, diuretics.

The invention is further illustrated and described with reference to the following specific example.

### EXAMPLE

This example describes a clinical trial involving the treatment of a small number of human patients with advanced chronic congestive heart failure. The patients had NYHA class III-IV chronic congestive heart failure, with a left ventricular ejection fraction (LVEF) of less than 40% (mean 22%) and a 6 minute walk distance of less than 300 m. All of the patients were receiving other CHF treatments, including ACE inhibitors, β-blockers, aldosterone antagonists, digoxin, diuretics.

Protocol:

Patients received 1 injection of treated blood on each of 2 consecutive days, followed by single injections at monthly intervals commencing 14 days after their first injection, for a period of 5 months. Each injection had a volume of 10 ml. Each individual treatment comprised the following steps:

1. Collection of 10 ml of a patient's own venous blood into 2 ml of sodium citrate for injection, USP. The sodium citrate was added to the sample to prevent the blood from coagulating during the treatment.

2. Transfer of the citrated blood sample to a sterile, disposable container.

3. Ex vivo treatment of the blood sample by simultaneous exposure to:
an elevated temperature of 42.5° C.,
a gas mixture of medical grade oxygen containing 14.5 μg/ml of ozone bubbled through the blood sample at a flow rate of 240 ml/min (at STP);
and ultraviolet light at a wavelength of 253.7 nm.
4. Transfer of the blood sample from the sterile disposable container to a sterile syringe.
5. Intramuscular injection of 10 ml of the treated blood sample into the gluteal muscle of the same patient, in most instances following a local anaesthetic (1 ml of 2% Novocain or equivalent) at the injection site.

The ex vivo treatment of the blood sample described in step (3) above was performed with an apparatus as generally described in U.S. Pat. No. 4,968,483 to Mueller et al. The blood sample was simultaneously exposed to all three stressors for a period of 3 minutes.

Patients were monitored for adverse events during each visit. As well, a post-treatment follow-up was conducted to monitor survival, hospitalizations, and significant adverse events.

The trial involved 73 advanced CHF patients. QT-c interval was measured in 35 of the patients, from electrocardiograms of the patients, at the start of the trial (baseline) and at the end (final), 20 of whom had received the treatment and 15 of whom had received placebo. Two patients in each group were on a drug (amiodarone) known to extend the QT interval. Patients receiving beta-blockers (50% of each group) had been on stable doses for at least three months prior to commencing the trial. In the remaining 38 patients, the QT-c interval could not be reliably measured at both time points because of atrial fibrillation, conduction abnormalities, the use of a pacemaker, or because the patient did not complete the study due to death.

A standard 12-lead ECG was obtained at the start and at the end of the trial. QTc was determined by averaging the QT interval from 3 consecutive beats in leads 11 and V4.

At the start of the trial (baseline), the average QT-c interval was almost the same for both the active treatment group and the placebo group at 0.460 seconds for the active treatment group and 0.459 seconds for the placebo group (which are above the normal range, which has an upper limit of 0.440 seconds). At the end of the study (follow-up), the average QT-c interval in the patients receiving placebo had worsened by almost 15 milliseconds to 0.474 seconds, Whereas the group receiving treatment according to the preferred embodiment of the invention had improved their average QT-c interval by 17 milliseconds, to 0.443 seconds. This difference of 30 milliseconds at the end of the study was interpreted to be statistically significant. Furthermore, the average QT-c interval after six months of treatment, 0.445 seconds, approached the upper limit of the normal range, of 0.440 seconds. These results are shown in Tables 1, 2 and 3.

TABLE 1

| | | Group | | |
|---|---|---|---|---|
| | | 10 ml | | |
| ANOVA 2p = 0.9766 | | Vasogen 991 | Saline | Both Groups |
| QTcB | N | 20 | 15 | 35 |
| (Baseline) | Mean | 459.73 | 459.17 | 459.49 |
| | Std Dev | 57.44 | 51.86 | 54.32 |
| | Min | 353.2 | 375.7 | 353.2 |
| | Max | 542.6 | 558.3 | 558.3 |

TABLE 2

| | | Group | | |
|---|---|---|---|---|
| | | 10 ml | | |
| ANOVA 2p = 0.0890 | | Vasogen 991 | Saline | Both Groups |
| QTcB | N | 20 | 15 | 35 |
| (Follow-up) | Mean | 443.37 | 473.88 | 456.44 |
| | Std Dev | 48.39 | 54.29 | 52.50 |
| | Min | 339.2 | 386.2 | 339.2 |
| | Max | 523.2 | 600.4 | 600.4 |

TABLE 3

| | | Group | | |
|---|---|---|---|---|
| | | 10 ml | | |
| ANOVA 2p = 0.0724 | | Vasogen 991 | Saline | Both Groups |
| QTcB | N | 20 | 15 | 35 |
| (Baseline) | Mean | −16.36 | 14.71 | −3.05 |
| | Std Dev | 49.97 | 47.69 | 50.74 |
| | Min | −123.7 | −70.7 | −123.7 |
| | Max | 45.0 | 79.8 | 79.8 |

When the results for the active treatment sub-group of patients having a QT-c interval at the start of the trial longer than the normal range (average 0.508 seconds, 10 patients) were isolated and compared with those for the corresponding sub-group of placebo patients (average 0.490 seconds, 9 patients) the reduction in QT-c interval for the treated patients was even more marked. The active treatment sub-group showed an average QT-c interval reduction after the course of treatment to 0.462 seconds, whereas the reduction achieved with the placebo sub-group was on average to 0.483 and not to be of statistical significance.

In addition, there was a marked difference in mortality rates between the placebo group (7 deaths, all cardiac related) and the active treatment group (1 death, not cardiac related), a difference that could well be explained by the improved QT-c interval in the treatment group.

The corresponding figures corrected with the Fridericia formula were, at the start of the trial (baseline), 0.447 seconds for the treatment group and 0.450 seconds for the placebo group; at the end of the trial (follow-up_{—}, 0.429 seconds for the treatment group and 0.463 seconds for the placebo group. These results are shown below in Tables 4, 5 and 6, and the mean values of the two groups are presented as bar graphs on the accompany Figure of drawings, where the QTc in milliseconds is plotted on the vertical axis.

TABLE 4

| | | Group | | |
|---|---|---|---|---|
| | | 10 ml | | |
| ANOVA 2p = 0.8620 | | Vasogen 991 | Saline | Both Groups |
| QTcF | N | 20 | 15 | 35 |
| (Baseline) | Mean | 447.31 | 450.45 | 448.66 |
| | Std Dev | 53.34 | 51.24 | 51.70 |
| | Min | 349.4 | 370.8 | 349.4 |
| | Max | 526.7 | 559.9 | 559.9 |

TABLE 5

| | | Group | | |
|---|---|---|---|---|
| | | 10 ml | | |
| ANOVA 2p = 0.0345 | | Vasogen 991 | Saline | Both Groups |
| QTcF | N | 20 | 15 | 35 |
| (Baseline) | Mean | 428.99 | 462.61 | 443.40 |
| | Std Dev | 44.64 | 44.61 | 47.09 |
| | Min | 342.1 | 381.0 | 342.1 |
| | Max | 505.7 | 572.3 | 572.3 |

TABLE 6

| | | Group | | |
|---|---|---|---|---|
| | | 10 ml | | |
| ANOVA 2p = 0.0692 | | Vasogen 991 | Saline | Both Groups |
| QTcF | N | 20 | 15 | 35 |
| (FUP-Base) | Mean | −16.32 | 12.16 | −5.26 |
| | Std Dev | 47.44 | 47.61 | 49.25 |
| | Min | −126.3 | −64.3 | −126.3 |
| | Max | 40.2 | 73.1 | 73.1 |

QT dispersion (QTd) was also determined from the same ECGs, by averaging the QT interval from 3 consecutive beats in each ECG lead and calculating the difference between the shortest and longest mean value.

QTd decreased in the treated group by 16 milliseconds during the study, while it increased by 19 milliseconds in the placebo group, showing a significant between-group difference at end of study (59.71±22.85 vs. 82.08±32.35 msec, ANOVA p=0.035).

Prolonged depolarisation, as well as heterogeneous repolarization (QT dispersion), in turn, contribute to arrhythmogenesis (Tomaselli et al, “Sudden cardiac death in heart failure. The rule of abnormal repolarization”, Circulation 1994; 90:2534-9). The observations that QTc and QTd increased in the placebo-treated patients but decreased in those receiving treatment by the process of the invention suggests a reversal of electrophysiologic remodelling in the treated patients. This may also be a marker for improved overall cardiac function.

These results indicate potential for the process of the invention to treat, or to exercise a preventative effect against the onset of, a wide variety of cardiac disorders associated with or manifesting themselves in prolonged QT intervals. These include ventricular arrhythmias such as torsade de pointes, a well as sudden death from heart disease. Moreover, sudden infant death syndrome (SIDS) has been associated with prolonged QT-c interval. Accordingly, the process of the present invention has potential in treatment of infants who exhibit prolonged QT-c intervals, to lessen the risk of their subsequently encountering SIDS.

Although the invention has been described with reference to specific preferred embodiments, it will be appreciated that many variations may be made to the invention without departing from the spirit or scope thereof. All such modifications are intended to be included within the scope of the following claims.

1. A process for treatment or prophylaxis of a mammalian cardiac disorder, the presence of or the susceptibility to which is detectable by observing a prolonged QT-c interval on an electrocardiogram of the patient, which comprises administering to a mammalian patient suffering therefrom or susceptible thereto an aliquot of compatible mammalian blood which has been extracorporeally subjected to oxidative stress and electromagnetic radiation.

2. The process of claim 1 wherein the disorder is cardiac instability.

3. The process of claim 1 wherein the disorder is arrhythmia.

4. The process of claim 1 wherein the patient is susceptible to sudden cardiac death.

5. The process of wherein the mammalian patient is an infant susceptible to sudden infant death syndrome.

6. The process of claim 1 wherein the mammalian patient, prior to administration of said blood aliquot, exhibits a QT-c interval of at least 0.440 seconds.

7. The process of claim 1 wherein the electromagnetic radiation is ultraviolet light.

8. The process of claim 7 wherein the oxidative stress is gaseous oxygen/ozone mixture, bubbled through the blood aliquot simultaneously with the subjection to ultraviolet light.

9. The process of claim 8 wherein the blood aliquot is maintained at temperatures within the range about 40-50 degrees C.

10. The process of claim 1 wherein the aliquot is autologous blood of volume from about 5-15 ml, and is administered to the patient intramuscularly.
